(19) United States
(12) Patent Application Publication
(10) Pub. No.: US 2020/0335182 A1
(43) Pub. Date: Oct. 22, 2020

(54) Method and apparatus for facilitating the binding of biological macromolecules with the use of gluing molecular agents with applications in RAS mutations and related conditions

(71) Applicant: Vince Grolmusz, Budapest (HU)

(72) Inventor: Vince Grolmusz, Budapest (HU)

(73) Assignee: Uratim Ltd., Budapest (HU)

(21) Appl. No.: 16/846,346

(22) Filed: Apr. 12, 2020

Related U.S. Application Data
(60) Provisional application No. 62/834,498, filed on Apr. 16, 2019.

(51) Int. Cl. G16C 20/50; G16B 5/30

(52) U.S. Cl. CPC G16C 20/50; G16B 5/30

(57) ABSTRACT

The binding of different biological macromolecules play an important role in numerous biological processes. In some diseases or abnormal conditions the binding is inhibited by some chemical or geometrical effects. The inhibited binding may cause serious diseases or conditions. The present invention discloses a molecular docking method, by which the binding may be enhanced, and the negative effects may be erased. The method can be applied as a novel framework in drug discovery and drug design in numerous settings, including, but not limited to mutations in the RAS protein, leading to human carcinomas.

### CROSS REFERENCE TO RELATED APPLICATIONS

This application claims priority from U.S. provisional patent application Ser. No. 62/834,498, filed Apr. 16, 2019.

### BACKGROUND OF INVENTION

Mutated genes are causing several serious illnesses, including different cancers. More than 600 cancer causing genes are listed in in the Catalogue of Somatic Mutations In Cancer, https://cancer.sanger.ac.uk/cosmic. Some of these mutations change the steric properties of the proteins, which modify their function. One important example is the RAS gene, whose mutations are found in more than 25% of human tumors. The mutations of RAS are connected to some of the hardest-to-treat, most lethal cancers [1].

The RAS protein was tagged “undruggable” until the most recent times, since it escaped the molecular docking efforts, because of the lack of the molecular docking cavities on its surface [2,3].

It is known from the prior art that the main reason for the oncogenic effects of the RAS is that they prevent the formation of the protein complex, consisting of the RAS and the GTPase-activating protein (GAP). The oncogene mutations have a steric structure that does not allow the RAS-GAP binding.

Here we disclose a general method for the enhancement of molecular binding of macromolecules, through the application of molecular docking to the specific conformations of those macromolecules.

Molecular docking is an algorithmic, computer-based molecular modelling approach, which computes the binding affinity or energy of two molecules, most frequently a macromolecule (or receptor) and a small, drug-like molecule, chosen from a high number of candidate molecules or a large computer based molecular database. Molecular docking is applied in the last two decades for finding new drug candidates by screening large databases of drug-like small molecules [4], facilitating virtual screening of potential drug molecules.
[1] Stephen, A. G., Esposito, D., Bagni, R. K., McCormick, F. Dragging RAS back in the ring, Cancer Cell 25, 272-281 (2014)
[2] Downward, J. RAS's cloak of invincibility slips at last? Cancer Cell 25, 5-6 (2014).
[3] Wang, W., Fang, G. & Rudolph, J. RAS inhibition via direct RAS binding—is there a path forward? Bioorg. Med. Chem. Lett. 22, 5766-5776 (2012).
[4] Scheich, C, Szabadka, Z, Vértessy, B., Pütter, V., Grolmusz, V., Schade, M.: Discovery of Novel MDR-Mycobacterium tuberculosis Inhibitor by New FRIGATE Computational Screen. PLoS ONE 6(12): e28428. doi:10.1371/journal.pone.0028428 (2011).

### SUMMARY OF INVENTION

In macromolecular interactions, the binding of macromolecules may be enhanced by small molecules, which glue the macromolecules together. The invention relates to a method of finding such small, drug-like molecules. First, the accurate three-dimensional structure of the macromolecules are determined or acquired from a database, for example, the Protein Data Bank. Let us denote molecule A and molecule B the two macromolecules with known 3 dimensional structure. Next, a close, artificial configuration of the molecules A and B is generated from the data of the bound A-B complex in a way that molecule A and molecule B is positioned in a non-contacting position, with a gap between them; the width of the gap corresponds to the size of the typical small molecules which are to be inserted in the gap, to bind to both molecules A and B. Next, by the use of a molecular modeling software or a molecular docking software, a library of small molecules are docked in the gap between molecules A and B. The best small molecules, which bind to both A and B, are identified by the scoring mechanism of the docking or molecular modeling program. This way the small molecules can be identified, which can glue molecule A to molecule B, and they can enhance the binding of A to B, and the bound A-B complex can have a beneficial biological property.

### BRIEF DESCRIPTION OF DRAWINGS

FIG. 1. schematically shows the macromolecule A (101) and macromolecule B(102) in the natural, healthy configuration: here the two macromolecules bind to each another.

FIG. 2. schematically shows the mutant macromolecule A (201, 203) and the macromolecule B (202) in the mutant case: the mutant A molecule has a steric anomaly (203), preventing the bound between the molecule A (201,203) and molecule B (202).

FIG. 3. schematically shows the small molecule (304), which connects, or “glues” the mutant macromolecule A (301,303) to macromolecule B (302); therefore, facilitating the bound between the mutant macromolecule A (301,303) and the macromolecule B (302) in the presence of the steric anomaly (303) in the mutant macromolecule A (301).

FIG. 4. is a flowchart, disclosing the main steps of the novel method of gluing molecules A and B.

### DESCRIPTION

Macromolecular interactions are important in numerous biological processes. In certain conditions, these interactions are inhibited by geometrical or chemical causes, and the inhibited interactions may lead to abnormal conditions or diseases.

One well-known example is the mutation of the RAS molecule. Here the RAS protein molecule, due to a mutation in a single amino acid, becomes unable to bind to the GAP (GTPase-activating) protein. The lack of binding initializes a cascade of biochemical events, which leads to uncontrolled cell division and, finally, cancer growth [Stephen, A. G., Esposito, D., Bagni, R. K., McCormick, F. Dragging RAS back in the ring, Cancer Cell 25, 272-281 (2014)]. RAS mutations are connected to several of the most lethal cancers today, including pancreatic ductal adenocarcinoma, colorectal adenocarcinoma, and lung adenocarcinoma.

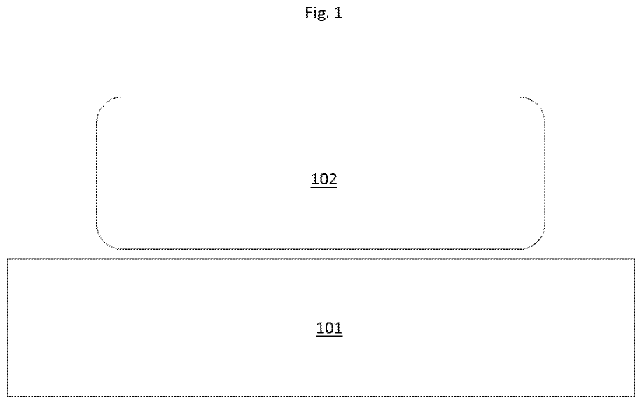

FIGS. 1,2 and 3 schematically describe the invention disclosed hereby in a general setting. Applying to the RAS-GAP binding enhancement, the figures can be translated to the specific application as follows:

FIG. 1. schematically shows the RAS molecule (101) and the GAP molecule (102) in the non-mutant (called “wild”) configuration: here the two proteins bind to each another.

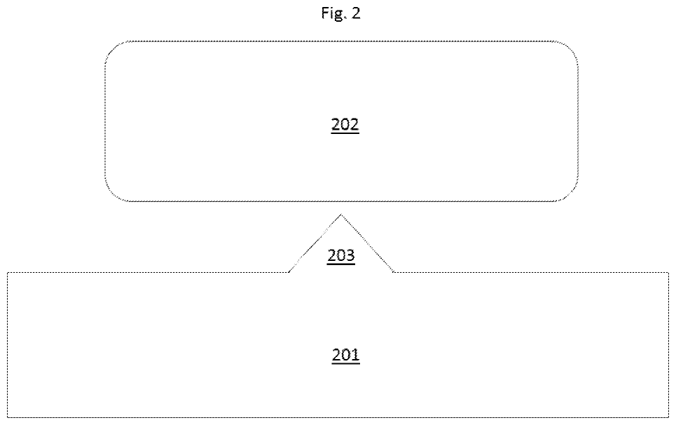

FIG. 2. schematically shows the mutant RAS molecule (201, 203) and the GAP molecule (202) in the mutant case: the mutant RAS molecule has a steric anomaly (203), preventing the bound between the RAS (201,203) and the GAP (202) molecules.

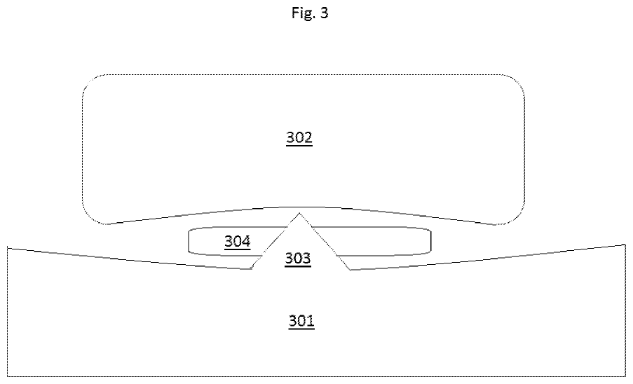

FIG. 3. schematically shows the small molecule (304), which connects, or “glues” the mutant RAS (301,303) to the GAP (302); therefore, facilitating the bound between the mutant RAS (301,303) and the GAP (302) in the presence of the steric anomaly (303) in the mutant RAS (301).

In the general setting, FIGS. 1,2 and 3 describe the method of finding gluing small molecules for enhancing the binding of macromolecules A and B.

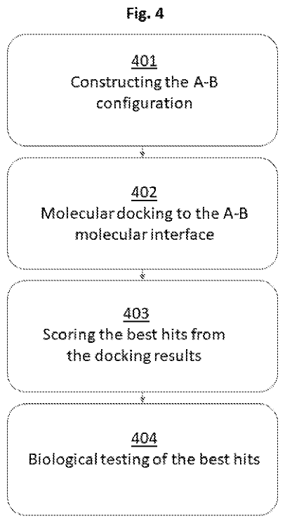

The invention is disclosed on FIG. 4 as a flowchart of the steps to be taken for finding small molecules, gluing molecules A and B.

Step 401 on FIG. 4 can be accomplished by starting from the bound A-B configuration (FIG. 1, the molecules 101 and 102), and substituting the mutated molecule 201-203 on FIG. 2, instead of 101, such that a gap is formed between molecules 201 and 202 where the width of the gap needs to be corresponded to the size of the small molecules 304 to be used for gluing 201 and 202 together, demonstrated on FIG. 3, by 301 and 302.

For the molecular docking—step 402—one may apply commercially available (Gold, AutoDock, Dock) or in-house constructed software solutions. The docking software needs a screening library of small molecules. For the library of the small molecules, one can apply public repositories, like the ZINC repository at the address http://zinc.docking.org.

The scoring of the docking results—step 403—are done by the scoring function in the docking software.

The best small molecules, which found in steps 401-403 need to be verified by biological effects in animal models or cell cultures; this is step 404 of the disclosure.

What is claimed is:

1. A method for enhancing the bound of two macromolecules, referred to as molecule A and molecule B by small molecules, acting as molecular glues, comprising the following steps:
the accurate three-dimensional structure of the macromolecules A and B are determined or acquired from a database,
a close, artificial configuration of the molecules A and B is generated from the data of the bound A-B complex in a way that molecule A and molecule B is positioned in a non-contacting position, with a gap between them; the width of the gap corresponds to the size of the typical small molecules which are to be inserted in the gap, to bind to both molecules A and B.
by the use of a molecular modeling software or a molecular docking software, a library of small molecules are docked in the gap between molecules A and B.
the best small molecules, which bind to both A and B, are identified by the scoring mechanism of the docking or molecular modeling program

2. A method of claim 1, wherein the macromolecules A and B are both proteins.

3. A method of claim 1, wherein macromolecule A is a protein and macromolecule B is a peptide.

4. A method of claim 5, wherein both macromolecules A and B are peptides.

5. A method of claim 1, wherein macromolecule A is a RAS protein and macromolecule B is a GAP (GTPase-activating) protein.

6. A method of claim 1, wherein macromolecule A is a protein, macromolecule B is a nucleic acid.

7. A method of claim 1, wherein the molecular docking software is an energy-optimizing molecular docking software.

8. A method of claim 1, wherein the molecular docking software is an incrementally optimizing molecular docking software.

9. A method of claim 1, wherein the small molecule binding is simulated by a molecular dynamics software;

10. A method of claim 1, wherein the small molecules for binding are chosen from peptide molecules;

11. A method of claim 1, wherein the artificial configuration of A and B is generated by moving the coordinates of molecule A in the opposite direction from molecule B by distance d, where d is proportional to the size of the small molecules, screened for binding to both A and B.

12. A method of claim 11, wherein the configurations of A and B are optimized after the move, described in claim referenced.

13. A method of claim 1, wherein the gap between the molecules A and B are generated in several widths, and the molecular docking is performed separately for each gap width.
